(19) United States
(12) Patent Application Publication
(10) Pub. No.: US 2022/0372462 A1
(43) Pub. Date: Nov. 24, 2022

(54) METHOD FOR COVALENT IMMOBILIZATION OF MOLECULAR COMPOUNDS

(71) Applicant: REGENHU AG, 1690 Villaz-St-Pierre (CH)

(72) Inventors: Marcela Bilek, New South Wales (AU); David McKenzie, New South Wales (AU); Seyedeh Khadijeh Alavi, New South Wales (AU); Charles Oliver Morgan Lotz, New South Wales (AU)

(21) Appl. No.: 17/767,210

(22) PCT Filed: Oct. 9, 2020
(86) PCT No.: PCT/EP2020/078463

§ 371 Date: Apr. 7, 2022

(30) Foreign Application Priority Data
Oct. 11, 2019 (CH) 01300/19

(51) Int. Cl. C12N 11/02; B05D 1/02; B05D 3/14; C07K 17/08

(52) U.S. Cl. CPC C12N 11/02; B05D 1/02; B05D 3/144; C07K 17/08; C12N 2533/52

(57) ABSTRACT

Disclosed herein is a method for covalent immobilization of molecular compounds on a substrate surface, comprising the steps: Providing a substrate surface; Treating the substrate surface with a plasma at atmospheric pressure, thereby generating an activated surface site; Exposing at least the activated surface site, or some fraction of the activated surface site, to molecular compounds, thereby establishing a covalent bond between the molecular compounds and the substrate surface.

### FIELD OF DISCLOSURE

The present invention lies in the field of covalent immobilization of molecular compounds to a substrate surface. In particular, it is directed to a method for covalent immobilization of molecular compounds on a substrate surface suitable for 3D printing as well as a substrate comprising a substrate surface with covalently immobilized molecular compounds obtained with such a method.

### BACKGROUND, PRIOR ART

3D Bioprinting is a variant of additive manufacturing in which cells are printed together with non-biological materials. 3D Bioprinting has a wide range of applications in the fields of medicine and tissue engineering. Such applications include in-vitro environments for culture of cells and tissues to be used for research and drug screening as well as potentially the creation of replacement tissues and functional human organs. For these applications, it is desirable to provide an environment suitable for the growth of desired cell types at specific locations on the surface of the printed object.

For optimal biochemical signaling to guide cells and tissue integration, molecular compounds that facilitate adhesion, differentiation and proliferation of the desired cell types must be bound to the surface and present their bioactive motifs to the cells. The molecular compounds on a device to be used in contact with protein containing solutions, such as cell culture media, or on an implanted device need to be covalently bonded to prevent displacement through protein exchange as observed in the Vroman effect (see Hirsh et al Langmuir 1980, 26, 14380-14388 and Vroman et al Blood 1980, 55, 156-159).

Chemical methods for covalent immobilization of molecular compounds are not convenient for use in 3D printers, because these methods rely on multiple wet processing steps that often involve long reaction times with reagents and solvents, which may be toxic and/or require removal by rinsing to avoid side reactions or other unwanted effects.

Dry plasma methods involving energetic ion bombardment that enable covalent immobilization of bioactive molecules in the absence of other reagents have been demonstrated (see for example Coad et al. Surface and Coatings Technology, 2013, 233, 169 ff.) but these methods require the use of plasma at low pressures which are incompatible with 3D printing.

### SUMMARY OF DISCLOSURE

Even though such dry plasma methods achieve rapid covalent immobilization and do not require chemical processing, these methods cannot be readily employed in a 3D printing environment as they require gas pressure below atmospheric pressure.

It is therefore an overall objective to advance the state of the art in the field of covalent immobilization of molecular compounds and preferably overcome the disadvantages in the prior art fully or partly.

Preferably, a method is provided which does not require low pressure, and includes a simpler overall experimental setup.

According to a first aspect, the overall objective is achieved by a method for covalent immobilization of molecular compounds on a substrate surface, comprising the steps:
a) Providing a substrate surface, wherein the substrate and the substrate surface preferably comprises or consists of a polymer material or a polymerizable material, preferably an organic and/or carbon containing polymer material;
b) Treating the substrate surface with a plasma at atmospheric pressure, thereby generating at least one activated surface site;
c) Exposing at least a portion of the at least one activated surface site to molecular compounds, thereby establishing a covalent bond between the molecular compounds and the substrate surface.

A plasma at atmospheric pressure is a plasma that is created in or exists in an environment at atmospheric pressure. Typically, the pressure is essentially 1 atmosphere. The use of such a plasma in the method according to the invention is beneficial, as it significantly reduces the complexity of the experimental setup. For example, the need for pumps, gas feeds and vacuum chambers is reduced or even eliminated for example when a dielectric barrier discharge with ambient air as the working gas is employed, which renders the process more efficient and less cost demanding. Due to the significantly higher pressure in atmospheric plasmas, as compared to low pressure plasmas used in energetic ion bombardment, the ion energies available in atmospheric discharges are much lower than those typical of low pressure discharges because of the significantly higher frequency of thermalizing collisions at atmospheric pressure. However, it has been surprisingly found that the surface of a polymer can indeed be activated for direct covalent attachment of molecular compounds by treatment with an atmospheric plasma. In typical embodiments, step b) is performed in the presence of oxygen or oxygen containing species. For example, step b) may be performed in air.

As the skilled person understands, an activated surface site is a surface site, which spontaneously forms covalent bonds with molecules subsequently brought into physical contact with the surface.

It is understood that it is not necessarily required in step b) that the whole substrate surface is treated with the plasma, but it is also possible that only one or more surface sites are treated with the plasma, thus generating one or more activated surface sites. This is useful for example for generating patterns on the substrate surface with an increased wettability. Correspondingly, in step c) not necessarily the whole activated surface site has to be exposed to molecular compounds, but it may also be possible to only expose a portion of the at least one activated surface site to molecular compounds, in particular by controlled dispensing.

A molecular compound as described herein may be a biomolecular compound, such as a cell, RNA, DNA, protein, oligonucleotide, aptamer, or may be a compound which can interact with a biomolecular compound, such as a hydrogel or a biologically active substance, such as an antibiotic.

In some typical embodiments, step c) is performed directly after step b). However, the generated active surface site still enables to establish a covalent bond with the molecular compounds after several hours, in particular after up to 24 h, or even up to a week.

In some embodiments, in step c), the whole activated surface site obtained in step b) is exposed to molecular compounds, for example by dipping the substrate surface into a solution containing the molecular compounds, or by spraying a solution of the molecular compounds onto the substrate surface, or any other method suitable therefor.

In other embodiments, in step c) only one or more portions, but not the whole activated surface site is exposed to molecular compounds. Preferably, the portion(s) of the activated surface site is (are) predetermined, such that a predefined pattern of molecular compounds is generated on the substrate surface. These embodiments are useful for example for generating patterns on the substrate surface with tailored wettability. Furthermore, it may be possible to expose a first portion of the activated surface site to a first molecular compound, a second portion of the activated surface site to a second molecular compound and/or a third portion of the activated surface site to a third molecular compound, etc. In doing so, the properties of the surface may be specifically adjusted at predetermined locations of the substrate surface. Exposing the portion(s) of the activated surface site is preferably performed by controlled dispensing of the molecular compounds. For example, the resolution may be controlled by varying droplet volumes and the surface tension of the solution containing the molecular compounds.

In some embodiments, the substrate surface comprises or consists of a polymer material, or a polymerizable material, which may optionally be deposited on the surface of a non-polymeric material such as a ceramic, semiconductor or metal. For example, a bare substrate that may be covered by the polymer or polymerizable material may comprise glass or titanium. Suitable polymer materials are known to the skilled person and include organic polymers, biopolymers and/or carbon based polymers. Typically, the polymer material or polymerizable material is configured for generating radicals or other reactive species upon plasma treatment under step b).

As the skilled person understands, a polymerizable material may typically be a monomer, which is configured to polymerize upon plasma treatment during step b) or may be a monomer which polymerizes upon irradiation with light of a specific wavelength, in particular UV light or which polymerizes after exposure to a chemical initiator. In some embodiments, the polymerizable material may be delivered via a nozzle of a plasma generation system.

In some embodiments, the at least one activated surface site at least temporarily comprises radical species, preferably oxygen centered radicals or other reactive species. These species can be coupled to specific moieties in molecular compounds, such as carbonyl, carboxyl, hydroxyl, amino, thiol, sulfate, phosphate, aryl, alkenyl, alkynyl groups or any other suitable group for establishing a covalent bond with such a radical, such as electrophilic or nucleophilic moieties.

In further embodiments, the polymer material or the polymerizable material is selected from a hydrocarbon polymer, such as polyethylene, polypropylene or polystyrene or precursors thereof, or from a heteroatom containing organic polymer, such as polytetrafluoroethylene, polyvinylchloride, polycaprolactam, polycaprolactone, poly(meth)acrylate, polyethers or polyesters or precursors thereof. As the skilled person understands, a precursor of a polymer material, such as polycaprolactone or polyacrylate, may be a suitable monomer, such as caprolactone or acrylate.

In some embodiments, the molecular compounds comprise cells, proteins, peptides, hydrogels, DNA, RNA, oligonucleotides, aptamers or antibiotics.

In further embodiments, step b) is performed for 0.001 s to 900 s, preferably 0.2 s to 900 s, 1 to 900 s, preferably 1 to 10 s at a particular surface site. It has been found that treating the particular surface site for only 2 s can be sufficient to provide a water contact angle of about 72° to 80°, as measured according to the “water contact angle test” as described herein. Noteworthy, the water contact angle is a direct indication for the wettability and thus the hydrophilicity of the particular surface site. Hydrophilic surfaces are desirable as they do not induce adverse changes in the aqueous solution conformations of the bound molecular compounds. Hydrophobic surfaces, in contrast, induce conformational changes or denaturation in adsorbed proteins which make the biomolecules lose their favorable biological activity in their interactions with cells and can even cause them to induce unfavorable immune responses of the host. Such unfavorable immune responses often result in the formation of fibrotic capsules that isolate the implants from the body, interfering with their function and eventually requiring revision surgery to remove the implants. These negative effects can be avoided by providing water contact angles of less than 80°. Noteworthy, the obtained contact angles are essentially maintained and stable over several days, even up to several weeks and beyond as indicated by the permanence or semi permanence of the reduction in contact angle indicated by the measurements.

In some embodiments, step b) may be repeated multiple times at a particular surface site, preferably 1 to 50 times, more preferably 1 to 20 times.

In further embodiments, the plasma is generated with a plasma generation system comprising a nozzle and a moveable single electrode or a movable double-electrode such that the electrode is movable along the substrate surface. When a double electrode is used, the distance of the respective electrodes may be selected between 0.1 and 300 mm, preferably between 10 and 40 mm. At greater distances, the breakdown voltage increases up to a point that is not conveniently attained by voltage supplies available. While the generation of activated surface sites is possible with both single electrodes and double electrodes, the use of double electrodes is advantageous for 3D bioprinting, because using a single electrode requires a lower earthed electrode on which the substrate is mounted. When a single electrode design is used the plasma discharge must be sustained by electric fields between that single electrode and an earthed external electrode, such as the build plate in a 3D bioprinter. A distance of 0.5 mm between the single electrode and the earthed external electrode provides an adequate plasma while with larger distances between the earthed electrode and the movable single electrode the electric field becomes weaker, reducing its surface modification capability. 3D bioprinting however not only requires a plasma for generating activated surfaces, but also structures of the printer, i.e. the additive manufacturing device and furthermore space of varying dimensions for the growing manufactured target structure. The distance between the target structure holder, i.e. the build plate, and the region of interest for modification must vary as the structure is constructed at increasing distances from the build plate. For a fixed voltage, this would result in a varying electric field leading to varying plasma discharge intensity, resulting in difficulty in controlling the surface modification. Limiting the distance to 0.5 mm severely restricts the space available for printer structures or the growing substrate. Using a movable double electrode has the advantage that both electrodes can be placed on a gas carrying tube and thus the space available for 3D printer structures and the growing target structure is greatly increased. In addition, several known single electrode designs are operated without gas flow, i.e. the gap between the movable single electrode and the stationary earthed electrode is filled with air. A movable electrode operated with a gas flow enables improved control of the surface modification outcomes such as the water contact angle and thus the wettability of the substrate. Furthermore, in designs without such gas flow, the reactive species generated in the plasma are only transported towards the surface by diffusion, thereby preventing printing complex structures, which are required for printing for example human bone substitutes and the like. In contrast, an electrode with a convective airflow can carry reactive species to the surface under treatment, and therefore the surface can have a complex geometry, including cavities and the like.

In some embodiments the electrode is operated at a voltage of 1 to 25 kV, preferably 3 to 12 kV and/or at a frequency of 1 kHz to 10 GHz, preferably at 20 kHz to 40 kHz. It should be noted that the singular use of the term “electrode” also refers to the two electrodes when a double electrode is employed. It has been observed that an increased voltage results in a reduced water contact angle. A double electrode allows for a stable electric field and hence a more stable discharge in the region beyond the nozzle where surface modifications are taking place. This is a significant advantage in 3D bioprinting where the distance between the build plate and the region of interest for modification varies.

In further embodiments, a distance of the electrode to the substrate surface is between 0.1 to 200 mm, preferably between 1 to 10 mm.

In some embodiments, step c) is performed for 5 minutes to 48 hours, preferably for 1 hour to 24 hours. In some embodiments, step c) may be performed by immersing the plasma treated substrate in a solution containing the molecular compounds, by 3D printing of the molecular compounds, or by depositing the molecular compounds by dropping or spraying.

In further embodiments, after step c), the surface is washed with a washing solution for removing any impurities or unbound molecular compounds. The washing solution may be any washing solution suitable for this purpose, such as distilled water, phosphate-buffered saline (PBS) buffer solution.

In some embodiments, a working gas is employed during step b), which is applied towards the substrate surface with a flow rate of at least 0.1 L/min. The working gas can carry reactive species to the surface under treatment, thereby allowing to employ even complex surface geometries. In particular embodiments, the flow rate may be 0.1 to 15 L/min, in particular. 0.1 to 12 L/min, preferably 0.5 to 12 L/min, in particular 1.5 to 12 L/min, more preferably 1.7 to 10 L/min, in particular 2.7 to 10 L/min. It has been found that decreasing the flow rate leads to an increase in the water contact angle. Thus, a flow rate of at least 0.1 L/min or higher is preferred. Typical workflow gases may be helium, argon, neon or xenon or oxygen enriched working gases, such as mixtures of water vapor and/or oxygen with helium, neon, argon, xenon, nitrogen or also pure oxygen. Polymerizable gases, such as acetylene, may also be included so as to deposit a polymeric surface coating during the activation process.

In preferred embodiments, the voltage with which the electrode is operated is 3 to 12 kV and the flow rate of the working gas is as described above, i.e. at least 0.1 L/min or higher, particularly 0.5 L/min or higher. Combining these two parameters has an additional beneficial effect on the water contact angle, as angles of 45 to 550 are obtained. Such contact angles are particularly beneficial, as outlined above, if the substrate surface is too hydrophobic (water contact angle of >90°), unfavorable conformational changes or denaturation of proteins or cells is observed. However, if the substrate is too hydrophilic (contact angle of <35°), interactions between cells immobilized on the substrate may be prevented.

In further embodiments, the voltage and/or the flowrate are chosen such that a water contact angle at the activated surface site of 35° to 80° is obtained, when measured according to the “contact angle test” as described herein.

In some embodiments, the molecular compounds are configured for adhesion of cells and wherein the method further comprises the application of cells to the covalently immobilized molecular compounds. As the skilled person understands, such particular molecular compounds may interact with and bind to the cells. For example, the molecular compounds may be natural or artificial proteins, which interact with or bind to the cellular membrane or with/to transmembrane proteins of the cell or antibodies, which are configured to bind to antigens of the cell. Examples for such proteins are extracellular matrix adhesion proteins such as elastin, tropoelastin, fibronectin, collagen and laminin and/or signaling molecules, such as cytokines, growth factors, metabolites and hormones, that regulate cell behavior by means of their interactions with receptors in the cell membrane. Such an embodiment enables the specific adhesion of cells through their membrane receptors to predefined positions, by providing active sites on the substrates, which then selectively covalently immobilize specific biomolecules configured for cell adhesion. It also enables the regulation of cell behavior.

In some embodiments, the cells may be applied by a 3D bioprinter. In such embodiments, the movable double electrode or single electrode may be an integral part of the 3D bioprinter. Consequently, the method according to any of the embodiments described herein can at least partially or completely be performed with a 3D bioprinter.

In some embodiments, the method further comprises the step of applying cells to the immobilized molecular compounds. The cells may be bound to the immobilized compounds by covalent bonds, or via other molecular interactions, such as ionic interactions, van-der-Waals-interactions, etc. In some embodiments, the molecular compounds are located in the cell membrane, i.e. may be transmembrane proteins.

In specific embodiments, the temperature of the substrate surface during step b) is between 0° C. to 500° C., preferably 15° C. to 350° C., more preferably 20° C. to 150° C.

In some embodiments, a predetermined pattern of immobilized molecular compounds is generated on the substrate surface by either
i) exposing only one or more predetermined portions in step c) to molecular compounds; or by
ii) treating only one or more predetermined sites of the substrate surface with the plasma in step b), thereby generating a predetermined pattern of at least one activated surface site.

Such embodiments are advantageous, as the molecular compounds can be immobilized in a predetermined pattern. For example, it may ultimately be possible to apply a molecular compound in a specific pattern, such that a predetermined 3 dimensional structure may be formed on the substrate surface. According to a further aspect, the overall objective is achieved by a substrate comprising a substrate surface with covalently immobilized molecular compounds obtained with the method according to any of the embodiments as described herein, wherein the water contact angle at an activated surface site of 35° to 80° is obtained, when measured according to the “contact angle test” as described herein.

### EXEMPLARY EMBODIMENTS

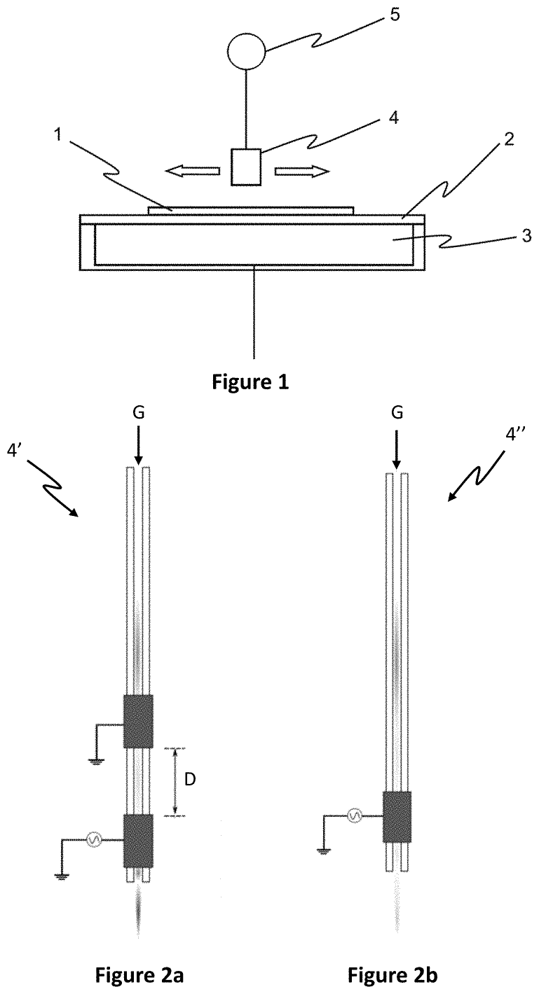

FIG. 1 shows a system for covalent immobilization of molecular compounds on a substrate comprising a substrate 1, a carrier 2, which may be made from sintered alumina, a grounded electrode 3 and a movable single or double electrode 4, which is connected to power supply 5. The rectangular electrode 4, which may have dimensions of 20 mm×20 mm×50 mm, was made of stainless steel. It could be scanned in one dimension along the length of the bottom electrode. The bottom electrode was a grounded steel plate, with dimensions 72 mm×160 mm, covered with sintered alumina (1 mm thick). The electrode configuration was such that the long side of the bottom electrode was parallel to the short side of the top electrode.

FIG. 2a shows a double electrode 4′, in which the electrodes are arranged such that they have a distance D between each other. The figure shows a configuration where the downstream electrode is powered and the upstream is ground, but the reverse can also be the case, and is preferred in some embodiments. FIG. 2b shows a single electrode 4″. Both electrodes surround a glass tube which is flushed with a working gas G flowing through it with controlled flow rate.

Three different substrate types have been used in the method according to the invention, namely a polytetrafluoroethylene (PTFE), a low density polyethylene (LDPE) and a polycaprolactone (PCL) substrate.

A representative example for a PTFE substrate is as follows: PTFE foil (50 μm thick) was cut into strips approximately 1.3 cm wide and about 6 cm long. Three strips were mounted side by side on the dielectric covered electrode and held down at the ends with glass microscope slides. Laboratory air at atmospheric pressure filled the chamber. The single or double electrode 4, 4′ or 4″ was scanned over the PTFE strips 10 times for a treatment time of 10 seconds, whilst being driven with a high-voltage, low-frequency power supply, operated at 27-29 W, with peak-to-peak voltage ii kV and frequency 22 kHz. The power was measured both from Lissajous figures (discharge voltage measured by a high-voltage probe vs. the voltage on a 100 pF current-integrating capacitor in series with the discharge) and by a real-time power measurement circuit constructed in-house. After the atmospheric plasma treatment, the strips were cut into samples with approximate dimensions 0.9 mm×1.3 mm and placed into wells of a 24 well plate for incubation in protein solution.

Sterile protein solutions of 50 μg/ml were prepared in phosphate buffered saline (PBS) for Bovine Serum Albumin (BSA) and in distilled water for tropoelastin. Aged (in laboratory atmosphere at room temperature) and freshly treated PTFE samples were incubated in 1 ml of protein solution. Unless stated otherwise, the samples were incubated for 4 days in the protein solution. Protein solutions were then aspirated and samples were rinsed twice for 10 min each in 1 ml fresh PBS. To determine the proportion of covalently immobilized protein, a sample from each otherwise identically treated pair of samples was then washed (3% SDS in distilled water) for 1 h at 80° C. After SDS washing, these samples were rinsed twice for 10 min each in 1 ml distilled water. All samples were dried prior to XPS measurement. Prior to use in cell experiments, samples (21 hours after plasma treatment) were incubated (4 days) in 50 μg/ml tropoelastin solutions made up in buffers with pH 7.4 (PBS) and pH 10 (NaH₂PO₄+Na₂HPO₄) and then rinsed twice for 10 minutes each in 1 ml fresh PBS.

Attachment of cells to a PTFE substrate was performed as follows: The plasma-untreated samples were sterilized by germicidal ultraviolet light irradiation for 20 min or in 70% ethanol (plasma-treated samples were regarded as sterile) and inserted into 24-well polystyrene cell culture plates (TPP, Switzerland; internal well diameter 15.4 mm). Then they were seeded with endothelial cells (ECs) that originated from bovine pulmonary artery (line CPAE ATCC CCL-209, Rockville, Mass.). Each well contained 30,000 cells (i.e., approximately 15,000 cells/cm²) suspended in 2 mL of the medium, i.e. minimum essential Eagle medium supplemented with 2 mM L-glutamine, Earle's balanced salt solution with 1.5 g/L sodium bicarbonate, 0.1 mM nonessential amino acids, 1.0 mM sodium pyruvate, and 20% of fetal bovine serum (FBS) (all chemicals from Sigma-Aldrich). The cells were cultured for 1, 3, 5, and 7 days at 37° C. in a humidified air atmosphere containing 5% CO₂. Three samples were used for each experimental group and time interval.

Water Contact Angle Test: Wettability of plasma treated surfaces by measuring the water contact angle using a Kruss DSA10-Mk2 contact angle goniometer by means of the sessile droplet method (see for example Clegg 2013, Contact Angle Made Easy pp. 4-10, 40-47). For the ageing tests, samples were stored in petri dishes after treatment within the ambient laboratory atmosphere at 23° C. for various periods of time before measurement. The water contact angles were determined as the average value of at least three measurements on equivalent samples.

The surface chemistry of untreated and plasma treated samples was analyzed using X-ray photoelectron spectroscopy (XPS). The survey and Cis high resolution spectra were obtained using a SPECS FlexMod spectrometer equipped with an MCD9 electron detector and a hemispherical analyzer (PHOIBOS 150). The X-ray monochromic source (Al Ka, hv=1486.7 eV) was operated at a power of 200 W (10 kV, 20 mA). The base pressure was always below 5×10⁻⁸ mbar, and the take-off angle was 90°. Spectra calibration and calculation of elemental composition were carried out using the CasaXPS software. The concentration of each element was calculated as an atomic percentage from the survey spectra. Contaminants such as sodium and chlorine from the buffer typically observed at levels of no more than a few percent were excluded from the calculation. A correction procedure was applied to eliminate the influence of adventitious carbon, which was observed in some samples exposed to laboratory atmosphere for long periods. In this procedure, where there was carbon measured in excess of what would be expected to come from the atmospheric plasma-treated surface and the BSA protein molecules (based on their known C/F ratio and C/N ratios respectively), the excess value was subtracted from the measured carbon atomic percentage, and the atomic percentages were scaled to total 100%. In all cases when this correction was applied the recalculated data came closer to the trend line, providing a level of confidence that the subtracted carbon was due to contamination.

Immobilization of two kinds of proteins was studied with LDPE and PCL substrates: Fibrinogen (FG) (50 μg/ml) and Bovine Serum Albumin (BSA) (66.6 μg/ml). FTIR-ATR spectral analysis was employed to investigate the protein attachment to the surfaces. LDPE film of 0.2 mm thickness was used as a substrate. LDPE film was chosen as its regions of IR absorbance do not overlap the absorption lines of the protein backbone (principally the amide peaks). FTIR-ATR spectra were measured using a Digilab FTS7000 FTIR spectrometer fitted with a multibounce ATR accessory (Harrick, USA) with a trapezium germanium crystal at an incidence angle of 45°. To obtain sufficient signal/noise ratio and resolution of spectral bands, 1000 scans were taken at a resolution of 4 cm⁻¹.

To investigate the immobilization of proteins on LDPE surfaces, 12 samples (10 mm×15 mm) were prepared for each test, including 6 plasma treated and 6 untreated samples for each protein. The treated area was a circle approximately 10 mm in diameter, formed by the spreading of the plasma plume over the sample surface. This treated area, as determined by the naked eye, was therefore slightly more than 50% of the total area of each sample.

For investigating FG immobilization, LDPE samples were treated for 10 s at a 13 mm distance from the nozzle using the one-electrode plasma jet design (FIG. 2b) with helium gas flow rate 9.5 L/min and an applied voltage amplitude of 7.5 kV. For BSA immobilization, the two-electrode design of plasma jet with a helium gas flow rate of 1.9 L/min and an applied voltage amplitude of 4.5 kV was used. The treatment time and sample distance from the nozzle were 5 s and 5 mm, respectively.

The 6 LDPE samples including 3 plasma-treated and 3 untreated samples were incubated in protein solutions. Every sample was immersed in 5 mL of protein solution overnight for 23 hours at 23° C. in the laboratory environment. The remaining 6 samples, including 3 plasma-treated and 3 untreated samples, were immersed individually in 5 ml of PBS buffer (PH 7.4) without protein for the same time and under the same conditions. These 6 samples were used as control samples in FTIR-ATR spectroscopy. For spectral analysis after incubation, all samples that had been in protein solution were washed in PBS buffer and then in Milli-Q water to remove buffer salts from their surface. The control samples in PBS buffer were also washed in Milli-Q water.

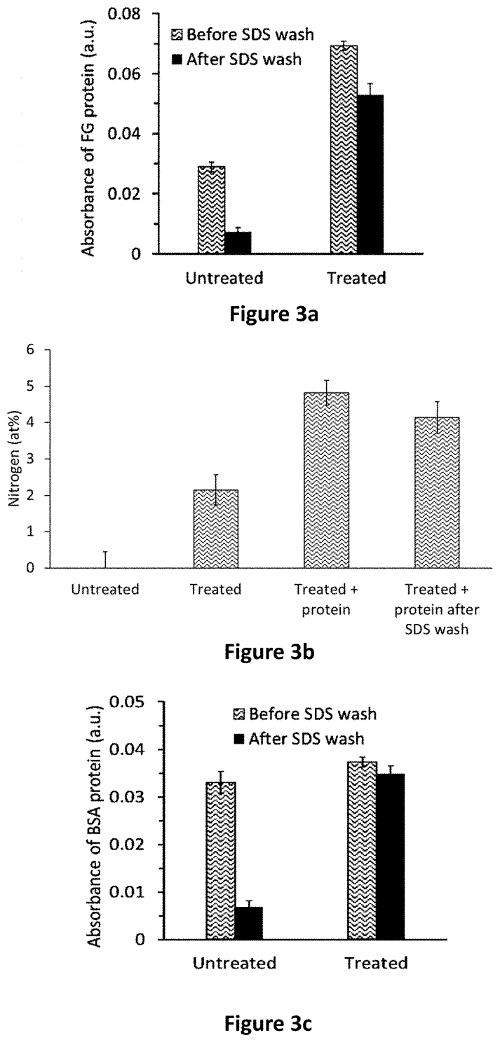

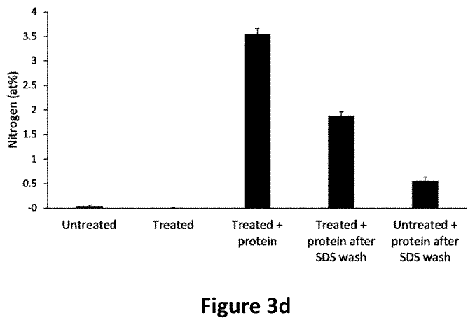

FIG. 3a: To confirm whether proteins were covalently immobilized onto the LDPE surfaces, FTIR-ATR spectral analysis after washing all 12 samples in 2% sodium dodecyl sulfate (SDS) solution was performed to quantify the amide protein backbone signal remaining (for the use of SDS to prove covalent attachment, see Bilek, McKenzie Biophysical Reviews 2010, 2, 55-65, which is incorporated by reference). An FTIR spectrum was obtained, from which the amounts of FG protein on the treated and untreated LDPE surface were calculated. As can be readily seen from FIG. 3a, most of the protein being present on the treated surface before washing (left column) is still present after washing (right column), while virtually all of the protein on the untreated surface (left column) is removed after washing (right column) as the FTIR signal is at the background level, thus indicating that FG indeed covalently binds to the treated surface but not to the untreated LDPE surface.

FIG. 3b: In the case of PCL, XPS measurements were performed for quantifying the nitrogen content for various differently treated PCL substrates. While untreated PCL contains no nitrogen, around 2% atomic concentration of nitrogen is present on the surface of the APPJ (atmospheric pressure plasma jet)-treated PCL. The nitrogen incorporation in the chemical structure of PCL after APPJ treatment is likely a result of reactions with nitrogen atoms from the ambient air. The nitrogen atomic concentration increases from 2% to approximately 4.8% upon protein attachment and decreases by only 0.8% after SDS washing, remaining significantly higher than that observed on the APPJ-treated surface. These results provide strong evidence that the BSA protein molecules are covalently attached to the plasma treated PCL surface.

FIG. 3c: To confirm whether proteins were covalently immobilized onto the LDPE surfaces another FTIR-ATR spectral analysis was carried out after a sodium dodecyl sulfate (SDS) solution wash. Treatment was conducted in the same way as for FIG. 3b. The samples were incubated for 22 hours with BSA at a concentration of 66.7 μg/mL (w/v) in PBS. Samples prepared under all conditions were washed in 5% (w/v) SDS solution at 70° C. for 1 hour. After the SDS washing, the samples were subject to a final Milli-Q water wash to remove residual SDS. SDS is a detergent capable of disrupting the forces responsible for physical adsorption and has no effect on covalent bonds. Amide peaks present in the FTIR-ATR spectra of samples incubated in protein and then properly washed with SDS provide evidence for covalent bonds between protein molecules and APPJ treated surfaces.

FIG. 3d: In another example, Bovine Serum Albumin (BSA) was attached to a Polydimethylsiloxane (PDMS) surface. PDMS was cut into 10×15 mm rectangles, 1 mm thick. PDMS was chosen as it is a polymer frequently used within microfluidic and biomedical devices. The APPJ surface treatments were carried out with a helium gas flow rate of 1.9 L/min, an applied voltage amplitude of 4.5 kV, and a frequency of 32.5 kHz. Sample distance from the nozzle was 5 mm. The APPJ was mounted in a 3D printer (FISun i3 Prusa) modified in-house. Treatment was conducted at a speed of 2,500 mm/min in lines with 5 mm distance center-to-center. BSA solution was made at a concentration of 66.6 μg/mL in PBS.

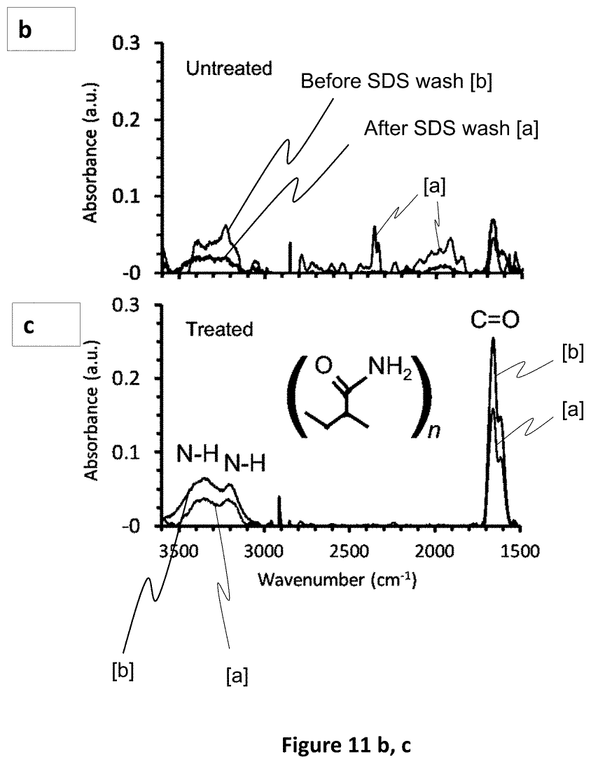

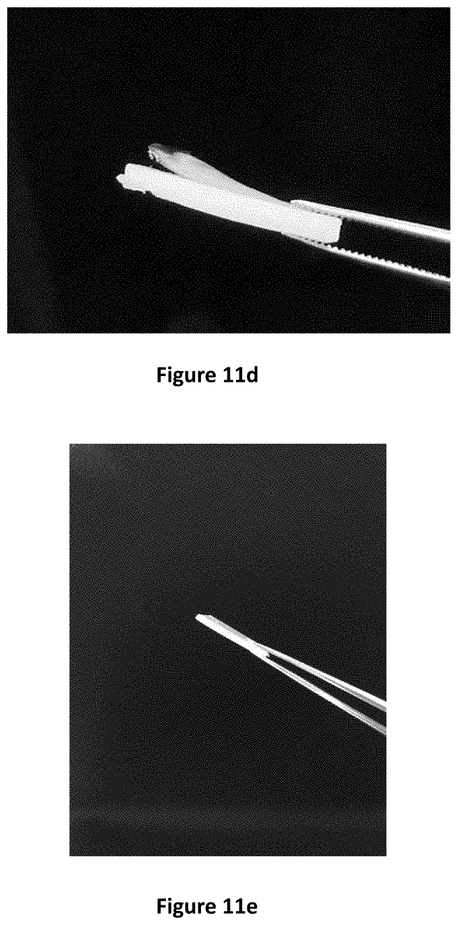

Samples were immersed in 5 mL of protein or buffer solution overnight for 25 hours at 23° C. in the laboratory environment. SDS washing was performed in the same manner as for BSA on LDPE (see for example FIG. 11). XPS survey, Nis and Cis high resolution spectra were obtained using an AXIS Nova (Kratos Analytical, Manchester, UK). The monochromic X-ray source used was Al Ka (hv=1486.7 eV). Spectral calibration and calculation of elemental composition were carried out using the Avantage software. When incubated without protein, untreated and APPJ-treated PDMS have atomic concentrations of nitrogen within the background error. When incubated with protein, the nitrogen concentration on APPJ-treated PDMS increases to 3.6%, and decreases to 1.9% after SDS washing, remaining significantly higher than that observed on the untreated surface after SDS washing. These results provide strong evidence that the BSA protein molecules are covalently attached to the plasma treated PDMS surface.

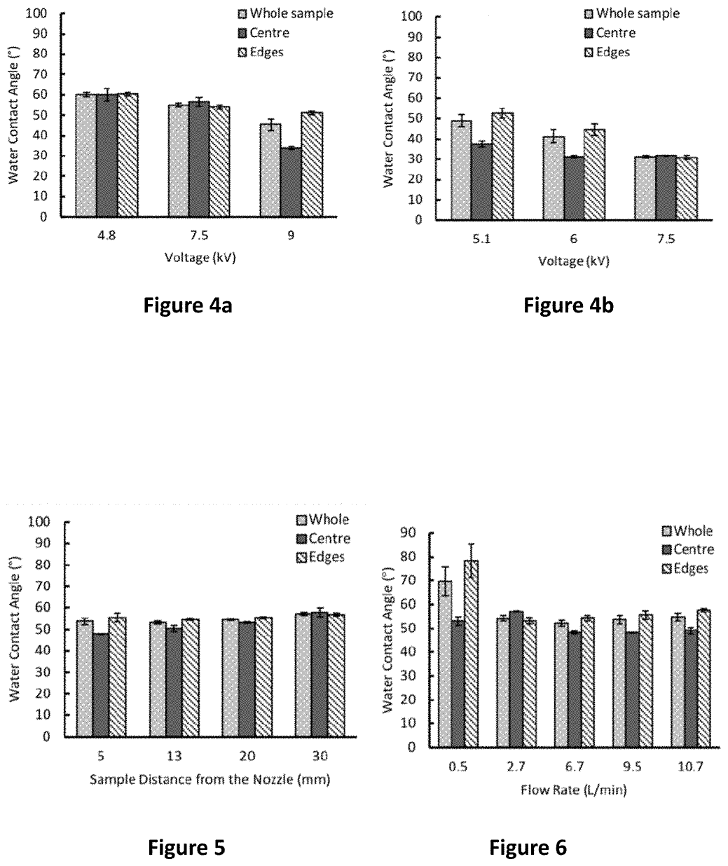

FIGS. 4a and 4b: To investigate the dependence of the applied voltage on the water contact angle for single (FIG. 4a) and double electrodes (FIG. 4b), LDPE samples were placed at a distance of 5 mm from the plasma nozzle at the tip of the electrode. In both cases, the contact angle decreases as the applied voltage increases, indicating a more intense modification. As the plasma plume spreads over the sample surface during treatment, every contact angle measurement was performed both directly under the nozzle (center) and at diametrically opposite positions at a radius of 3 mm (edges). The average values of all measurements are labelled as “whole sample”. The flow rate of the working gas (helium) for the single electrode was set to 9.5 L/min (FIG. 4a) and for the double electrode to 1.9 L/min (FIG. 4b). The treatment time for both experiments was 5 s.

FIG. 5: The influence of the distance between the electrode, respectively the tip of the glass tube nozzle which carries the electrode, to the LDPE substrate surface is shown in FIG. 5. The applied voltage was 7.5 kV at a helium gas flow rate of 9.5 L/min and the sample was treated for 10 s. As the distance increased, hydrophilicity reduced, indicating a less intense, but more uniform plasma treatment. As the plasma plume spreads over the sample surface during treatment, every contact angle measurement was performed both directly under the nozzle (center) and at diametrically opposite positions at a radius of 3 mm (edges). The average values of all measurements are labelled as “whole”.

FIG. 6: The effect of the gas flow rate on the contact angle is shown in FIG. 6. An LDPE substrate was treated with an atmospheric plasma from a single electrode. As the flow rate increases the contact angle decreases slightly in the center. However, in the interest of conserving helium, operation around a flow rate of 2.7 L/min may in general be preferred. Also, a remarkable difference exists between the contact angles at the center points and side points at 0.5 L/min flow rate. It shows that at very low gas flow rates the effective activated surface site is smaller than for the higher flow rates. As the plasma plume spreads over the sample surface during treatment, every contact angle measurement was performed both directly under the nozzle (center) and at diametrically opposite positions at a radius of 3 mm (edges). The average values of all measurements are labelled as “whole”.

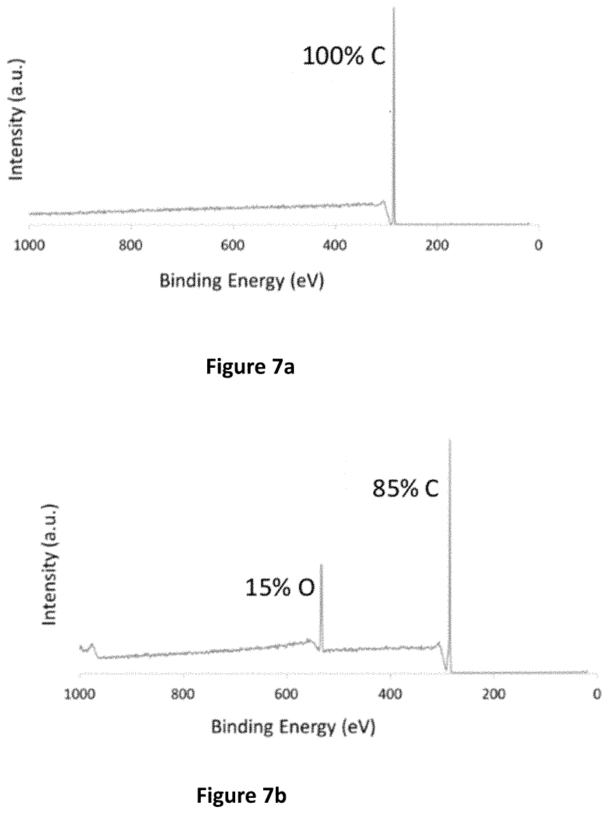

FIGS. 7a and 7b: XPS survey spectra of untreated and APPJ treated LDPE are shown in FIGS. 7a and 7b, respectively. Carbon atomic concentration decreases from 100% for untreated LDPE to 85% for APPJ treated surface, while that of oxygen increases from 0% to 15%. The increase of oxygen atomic concentration is due to surface oxidation induced by APPJ treatment.

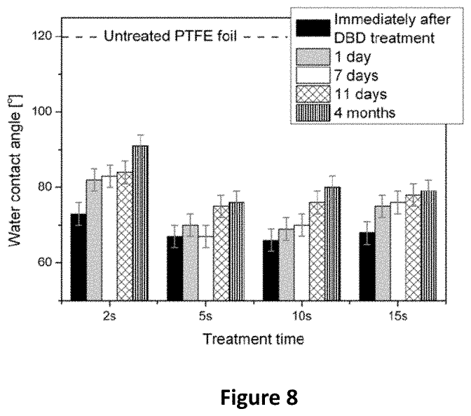

FIG. 8: The effect of the treatment time on a PTFE substrate on the contact angle is shown in FIG. 7. As can be readily seen, these measurements indicate a significant increase in wettability of the PTFE after atmospheric plasma treatment. The treatment was performed on PTFE foils at atmospheric pressure, 30 W power and 1.5 mm gap between electrodes. Each sample was measured immediately after treatment and then after 1 day, 7 days, ii days and 4 months. The water contact angle drops from 120 degrees to about 700 with the atmospheric plasma treatment, stabilizing at a slightly higher value around 80° after prolonged exposure to laboratory atmosphere, for all treatment times greater than or equal to 5 seconds.

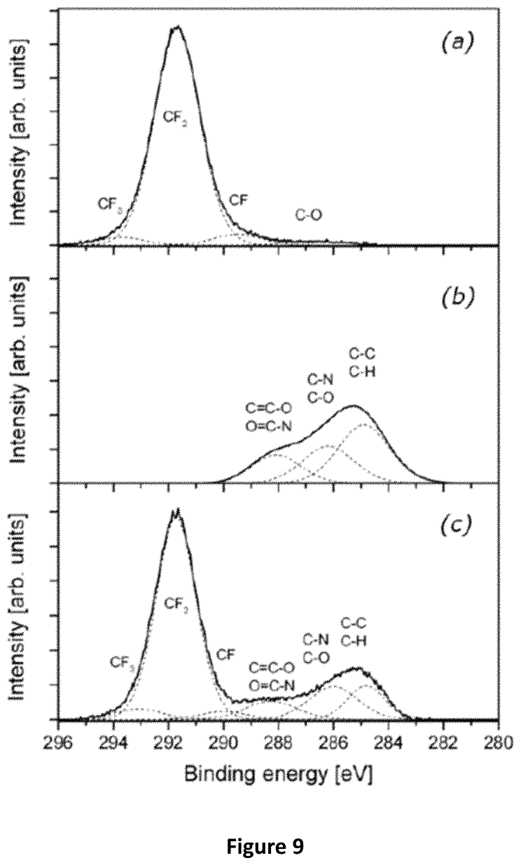

FIG. 9: XPS measurements of (a) PTFE substrate treated after 10 s plasma treatment (Peak fitting resolved peaks corresponding to CF₃, CF₂, CF and C—O groups at binding energies of 293.5, 291.7, 289.5 and 286.7 eV, respectively. The CF and C—O peaks are not present in the spectra of the untreated PTFE foil and only appear after atmospheric plasma surface treatment), (b) High-resolution Cis peak from a layer of dried BSA protein, thick enough to inhibit detection of Si peaks from the underlying substrate, (c) High-resolution Cis peak from an atmospheric plasma treated PTFE sample after 24 hours of incubation with BSA and subsequent SDS washing.

TABLE 1
Composition of untreated and atmospheric plasma-treated PTFE
foils showing the appearance of a small amount of oxygen upon
plasma surface treatment. Further changes in composition after
aging in laboratory atmosphere are small and are not significant
given the accuracy of the measurement (0.3 at % limit of sensitivity).
Adsorption of BSA protein on the surface decreases the fluorine
signal whilst increasing the signals of C, O and N. The amount
of protein adsorbed according to these increases is significantly
greater on the plasma treated foil than on the untreated foil.
Also noteworthy is the fact that the protein is completely removed
from the untreated foil after SDS detergent washing whilst much
of it remains on the plasma treated foils despite rigorous SDS
washing, as indicated by the retention of most of the N. *indicates
that the missing at % is made up of Na, Cl, S and P from buffer
salts that remain on the surface. These are completely removed
by the SDS wash.

| | C₁ₛ | F₁ₛ | O₁ₛ | N |
|---|---|---|---|---|
| | (%) | (%) | (%) | (%) |
| Untreated PTFE foil | 33 | 67 | 0 | 0 |
| 10s atmospheric plasma | 31 | 67.2 | 1.8 | 0 |
| 10s atmospheric plasma after 11 days aging | 31.2 | 67.8 | 0.9 | 0.2 |
| in air | | | | |
| Untreated PTFE after protein and rinse* | 33.2 | 64.1 | 1.5 | 1.0 |
| Untreated PTFE after protein and SDS | 32.3 | 66.7 | 0.8 | 0.3 |
| 10 s atmospheric plasma after protein and | 39.2 | 39.1 | 13.8 | 4.4 |
| rinse* | | | | |
| 10 s atmospheric plasma after protein and | 43.0 | 47.0 | 6.0 | 3.8 |
| SDS | | | | |

Further evidence for the retention of protein on the surface is provided by the presence of a nitrogen peak in the survey scan. Table 1 shows XPS composition data from elemental survey scans for representative samples that were incubated in BSA solution and then subjected to buffer and/or SDS detergent washing. The presence of BSA protein on the surface is revealed by the appearance of a nitrogen peak, a decrease in the fluorine peak intensity and increases in the intensities of C and O. The untreated PTFE is very hydrophobic, so adsorbed protein molecules unfold, exposing normally hidden hydrophobic residues to the surface and binding through hydrophobic interactions. The nitrogen signal is reduced to background levels indicating that protein is virtually all removed from the untreated surface by SDS washing. The surprising feature of the adsorbed layer on the plasma treated surface is that most of it is resistant to rigorous SDS washing, as indicated by a residual nitrogen signal of 3.8%. SDS is a detergent that is used to unfold proteins and to remove physically adsorbed proteins from surfaces. The SDS cleaning procedure has been extensively used as a test of covalent attachment to surfaces. These results indicate that a significant fraction of the surface adsorbed protein is covalently immobilized on the plasma-treated PTFE surface.

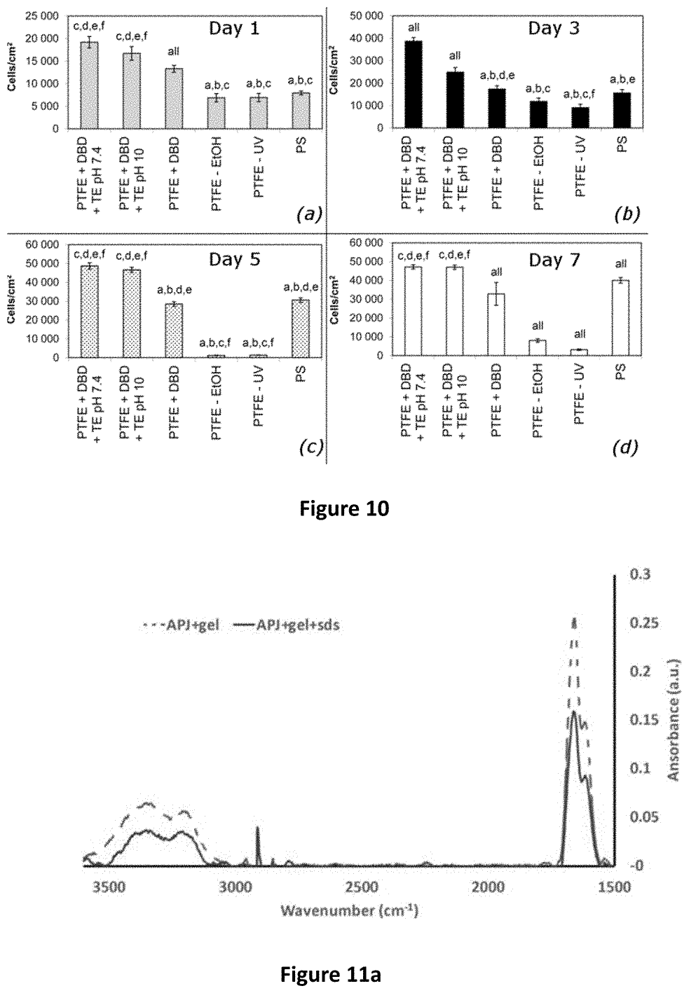

FIG. 10: To investigate the response of cells towards a substrate surface with molecular compounds immobilized, cell experiments were conducted as follows: (a) plasma-treated PTFE foil with tropoelastin immobilized at pH 7.4; (b) plasma-treated PTFE foil with tropoelastin immobilized at pH 10; (c) plasma-treated PTFE foil only; (d) PTFE foil sterilized in 70% ethanol; (e) PTFE foil sterilized in UV; (f) the bottom of standard polystyrene cell culture dishes (TCP) were used as controls. For cell experiments, bovine endothelial cells were used, and tropoelastin was immobilized on the treated PTFE samples 21 hours after atmospheric plasma treatment. On day 1 after seeding, the cell numbers on plasma-treated PTFE foils covered with tropoelastin at different pH were similar (19,200±1,300 and 16,700±1,500 cells/cm²). Moreover, the cells on these types of material differed in number from atmospheric plasma-treated PTFE foils without coverage (13,300±800 cells/cm²), pure PTFE foils sterilized by 70% ethanol or by UV (6,900±900 and 6,900±900 cells/cm², respectively), and control (the bottom of standard polystyrene cell culture dishes) (7,900±400 cells/cm²) (FIG. 10). Cells on atmospheric plasma-treated PTFE foils coated with tropoelastin (in pH 7.4 or pH 10) reached statistically higher population densities than on the other materials tested and maintained that trend during the entire experiment. Moreover, their spreading was better developed than on pure PTFE foils. The well-developed spreading area is considered a crucial factor for the appropriate attachment of the cells and leads to increased proliferation. In the experiment, the superior spreading is attributed to the presence of specific domains of the tropoelastin molecule recognized by cell adhesion receptors.

The adhesion and subsequent growth of anchorage-dependent cells (including endothelial cells) on artificial materials are mediated by extracellular matrix molecules (including elastin and its precursor tropoelastin) attached to the material surface. Specific bioactive sites in these molecules, usually specific amino-acid sequences, are recognized with cell adhesion receptors. For example, the sequence VAPG (Val-Ala-Pro-Gly) in elastin molecules is recognized by non-integrin adhesion receptors on vascular smooth muscle cells. In addition, vascular endothelial cells can bind elastin and tropoelastin by a cell membrane complex with a major glycoprotein component of 120 kDa, designated as elastonectin, by alpha v beta 3 integrins and also by alpha 9 beta 1 integrins, which can explain the highest initial adhesion and subsequent growth of endothelial cells on tropoelastin-covered PTFE. Also on plasma-treated PTFE, the adhesion and growth of endothelial cells were relatively good. This was most likely due to improved adsorption of the cell adhesion-mediating molecules fibronectin and vitronectin from the serum supplement of the cell culture medium to the material. It is believed that on substrates with a higher hydrophilicity, these molecules are adsorbed in a more physiological, flexible conformation, enabling a better recognition of specific bioactive sites in these molecules (namely, the amino acid sequences REDV and RGD) by cell adhesion receptors. Accordingly, on untreated and highly hydrophobic PTFE, cell adhesion and subsequent growth were poor, and from day 3 after seeding, the cell number decreased.

FIG. 11a-c: In a further example, it was tested whether a hydrogel may also be covalently attached to a substrate surface using the method according to the invention. LDPE was chosen as a first test polymer to be used with an acrylamide hydrogel. The condition without treatment displayed the characteristic peaks of the hydrogel upon FTIR-ATR measurement before SDS washing, but the peaks were not present at all after washing (FIG. 11b). However, the APPJ-treated surface retained a layer of the hydrogel after SDS washing, as observed in FIGS. 11a and 11c. It is evident that APPJ treatment is able to covalently attach hydrogel macromolecules to hard polymeric substrates. The hydrogel precursor used for attachment was acrylamide: N, N′-Methylene-bis-acrylamide monomer (29:1) at a concentration of 10% (w/v) in Milli-Q water without the addition of initiators for polymerization. The solution was left to degas for 15 minutes under a fume hood and then 15 minutes with high-purity argon going through the solution at a flow rate sufficient to cause bubbles in order to remove oxygen, which hinders the polymerization process. The 10×15 mm LDPE samples were then added to the solution. The acrylamide solution was placed in a heat bath at 80° C. for 50 minutes while the degassing with argon continued. Hydrogel coated samples were then washed 3 times in jars of Milli-Q water before drying in air and FTIR-ATR spectral analysis. Then 5% SDS at 70° C. for 1 hour was used for the subsequent wash. Baseline corrections were carried out on spectra for clearer display.

FIGS. 11d and e: In another example, adhesion of Gelatin Methacrylate (Gelma) on a PCL surface. Gelma solution was prepared by dissolving 10% (w/v) Gelma and 0.2% (w/v) Irgacure 2959 (2-Hydroxy-4_′-(2-hydroxyethoxy)-2-methylpropiophenone) photoinitiator in PBS. Dissolution was assisted by heating at 50 C. Gelma and PCL were chosen because they are both highly biocompatible, and therefore particularly relevant to tissue engineering applications. 200 μL of solution was added to each 10×15 mm PCL sample. Samples were then UV polymerized for 15 minutes with a wavelength of 365 nm. When the samples were dried for FTIR-ATR measurement, the Gelma peeled entirely off the untreated samples (FIG. 11d). This indicates weak adhesion. However, the dry Gelma did not peel off the treated samples, even after subsequent SDS washing (5%, 40 C) and drying (FIG. 11e). Therefore, the adhesion between Gelma and APPJ treated PCL is significantly stronger. For all treated samples before and after SDS washing, the characteristic peaks of the PCL substrate were not visible in the spectra because the layer of hydrogel was thicker than the penetration depth of the FTIR-ATR sampling radiation. This thickness suggests that the cross-linked hydrogel network itself is attached, rather than just a monolayer of the hydrogel monomer. Therefore, the thickness of hydrogel layers attached to hard polymeric substrates can be modified for a wide variety of applications.

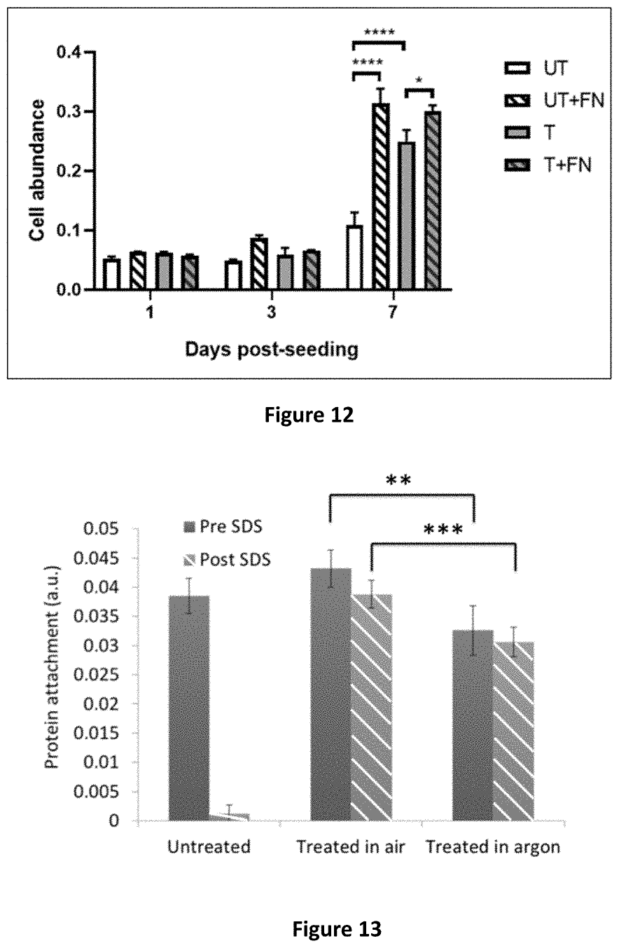

FIG. 12: A comparison of cell proliferation on (i) an untreated LDPE surface, (ii) an untreated LDPE surface with fibronectin, (iii) a treated LDPE surface and (iv) a treated LDPE surface with fibronectin revealed that the treated surface (T) promotes significantly higher cell proliferation that the untreated surface (UT). The addition of fibronectin increased cell proliferation for both untreated (UT+FN) and treated surfaces (T+FN). Although the untreated and treated samples both bind fibronectin sufficiently to promote cell attachment and proliferation in this experiment, the covalent attachment afforded by the plasma treatment confers significant advantages over physical adsorption to the untreated sample. Covalent binding of fibronectin ensures that it is robustly attached and prevents it being removed by various washing steps that are often required in applications. Covalent binding also prevents removal through the dynamic exchange with other proteins that occurs readily in physiological environments. When there is no protein attached by pre-incubation, the cell proliferation is promoted on the treated surfaces by covalent immobilization of serum proteins from the media, which creates a more biologically favorable substrate for cell growth. The hydrophilic nature of the treated surface is beneficial for preserving the native conformation and therefore the function of the covalently attached molecules. This is important for promoting cell attachment and proliferation because protein unfolded by interactions with a hydrophobic surface often elicits unfavorable cell responses. Untreated polyethylene has a water contact angle of approximately 100° degrees, while water contact angles of the plasma treated polyethylene are as low as 350 plateauing to approximately 550 after 3 days. The optimal range for biocompatibility is 35° to 80°. Hence, the plasma treatment brings the surface from hydrophobic down to an optimal range.

The experiment was conducted as follows: The low density polyethylene (LDPE) samples were cut to 6×8 mm rectangles of 0.2 mm thickness. The treatment was conducted at atmospheric pressure for 5 seconds using a helium flow of 1.9 L/min and a peak-to-peak voltage of 9.0 kV at a frequency of 32 kHz. There was a 5 mm separation between the APPJ nozzle and the sample surface. After UV sterilization for 30 min, samples were incubated in solutions of phosphate buffer solution (PBS), with and without fibronectin protein at a concentration of 4 μg/mL. They were incubated overnight at a temperature of 3-6° C. After incubation, samples were washed with PBS to remove excess unbound protein. Samples were seeded with human dermal fibroblasts at a density of 5000 cells/cm² in Dulbecco's Modified Eagle Media with 10% (v/v) fetal bovine serum. Media was changed every 2 days. At 1, 3 and 7 days post-seeding, cells were fixed by incubating the samples in 3% (v/v) formaldehyde at room temperature for 20 min. Cells were stained with 0.1% (w/v) crystal violet in 0.2 M 2-(N-morpholino) ethanesulfonic acid (MES) buffer for 1 hr at room temperature, then washed with reverse osmosis water to remove excess stain. Samples were imaged under bright-field microscopy. Cell abundance was quantified by solubilizing the crystal violet stain with 10% (v/v) acetic acid. Sample absorbance was read at 570 nm. Three equivalent samples were used for each condition. Errors displayed are standard error of the mean. The effects of the various substrates on cell proliferation were statistically compared with two-way analysis of variance (ANOVA).

FIG. 13: In order to investigate the role of ambient air during step b), surface treatment of a LDPE substrate under an air atmosphere was compared with treatment of a LDPE substrate under an argon atmosphere. As can be seen from FIG. 13, polyethylene samples treated in an air environment achieve greater covalent attachment of protein than those treated in a predominantly argon environment (**p<0.01 and ***p<0.001). The untreated samples had similar protein attachment to both types of treated samples before the SDS wash. However, effectively all the protein was detached during the SDS washing. SDS is a detergent that disrupts physical bonds whilst leaving covalent bonds intact. The fact that reducing the percentage of ambient air reduces the degree of covalent attachment supports the hypothesis that the constituents of air increase covalent attachment of molecular compounds at the treated samples.

The experiment was conducted as follows: A vacuum chamber system was constructed for the treatment of samples with the atmospheric pressure plasma jet (APPJ) in the presence of ambient gases of controlled composition. The chamber was pumped down to pressures below 7.0×10⁻² Torr before the ambient gas was introduced at a flow rate of 4.7 L/min. A pressure valve allowed excess gas to be released, once the chamber reached atmospheric pressure. The ambient gases, used separately in the following experiments, were argon and air, while the APPJ treatment gas was helium. When the ambient gas is not air, the residual air content can be calculated as the sum of the base pressure and the leak rate (measured as rate of increase of pressure after closing the pump valve and before inlet of any gas) multiplied by the time between closing the pump valve and reaching atmospheric pressure with the ambient gas introduced. In this case, the residual air content was about 0.03%. The samples treated were low density polyethylene (LDPE), cut to 10 mm×15 mm rectangles of thickness 0.2 mm. Treatment was conducted at atmospheric pressure for 10 seconds using a helium flow of 8.1 L/min, a peak-to-peak voltage of approximately 9.0 kV and at a frequency of 36 kHz. There was a 5 mm separation between the APPJ nozzle and the sample surface. Treated samples were then transferred from the chamber to the incubation solution as quickly as possible. This transfer process required approximately ten seconds. The incubation solutions were phosphate buffer solution (PBS) with and without BSA protein at a concentration of 333.3 μg/mL. The samples were incubated for 22 to 24 hours in the laboratory environment at 23° C. The protein on the surface was detected using Fourier transform infra-red (FTIR) spectroscopy equipped with an attenuated total reflection (ATR) crystal. The IR probe beam was coupled to the sample surface via the evanescent field at the crystal interface to improve surface sensitivity. FIG. 13 indicates the protein attached on each sample shown by the average of the amide I peak intensities measured from six equivalent samples. Errors displayed are the sum of the spectral noise and the standard error of the mean. Paired t tests were performed to compare the data from the various types of samples.

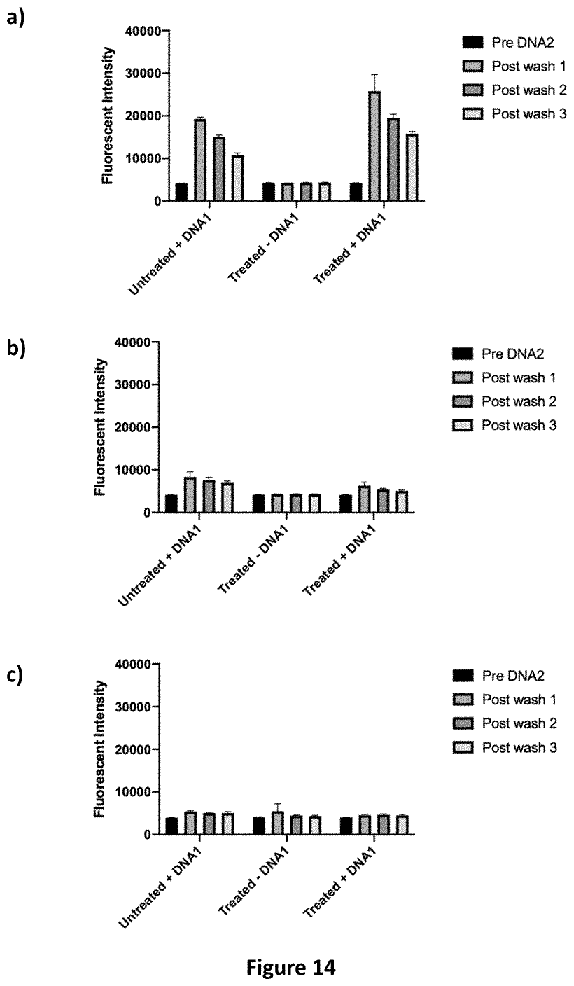

FIG. 14: In this example, DNA was bound to a LDPE sample. In addition, the influence of the pH at which the samples were incubated was examined. Samples incubated at pH 3 (FIG. 14a) have the highest fluorescent intensity, followed by pH 5 (FIG. 14b), and then pH 7 (FIG. 14c). As the pH drops, the number of hydrogen ions increases, and the higher the positive charge density in the solution. DNA strands within that solution also show an increase in positive charge density as pH drops. Therefore, greater fluorescent intensity at lower pH indicates that biomolecules have a higher rate of attachment when they are more positively charged. This rate of attachment implies that the treated surfaces may in fact be negatively charged. Negative charges on APPJ-treated surfaces support the observation that treatment introduces reactive oxygen groups to surfaces. The ‘Treated−DNA1’ conditions measured low fluorescent intensity for all pH, indicating that fluorescence detected was a result of DNA2 hybridizing with DNA1, as expected. In pH 3 (FIG. 14a), the ‘Treated+DNA1’ condition measured significantly higher fluorescent intensity than the ‘Untreated+DNA1,’ with a p<0.0001. This shows that the treated LDPE was able to bind the DNA significantly more strongly. The LDPE samples for this example were cut into 5×10 mm rectangles. The APPJ surface treatments were carried out with a helium gas flow rate of 1.9 L/min, an applied voltage amplitude of 4.5 kV, and a frequency of 32.5 kHz. Sample distance from the nozzle was 5 mm. The APPJ was mounted in a 3D printer (FISun i3 Prusa) modified in-house. Treatment was conducted at a speed of 2,500 mm/min in lines with 5 mm distance center-to-center. Samples were incubated for 1 hour in 160 μL of AAAAAAAAAAAAAAAAAAAAGCTCTGCAATCAACTTATCCC, referred to as ‘DNA1’, at a concentration of 2 μM in 10 mM pH 3 or pH 5 citric acid/sodium citrate buffer solution, or pH 7 Na2HPO4/NaH2PO4 buffer solution. All samples were then incubated in 10 mM PBS for 1 hour to block any remaining binding sites. Excess biomolecules were removed with a wash in 2% SDS at 25 C for 1 minute using 200 μL per sample and vortex shaking and repeated three times. All samples were then rinsed in PBS before 1 hour incubation in GGGATAAGTTGATTGCAGAGC with Alexa647, referred to as ‘DNA2,’ at a concentration of 0.8 μM in 2 mM MgCl2, 1×TE, 1% BSA, and 0.6% SDS. Finally, samples were exposed to a multi-step washing procedure at room temperature to remove weakly bound biomolecules. After another rinse in PBS, Wash 1 involved 0.1% SDS in saline sodium citrate (SSC). Wash 2 was the same again. Wash 3 involved 0.5% Tween20 in SSC before a PBS rinse. All biomolecular incubation was conducted at either pH3, 5, or 7, depending on the condition. Measurements were made in 24 well microplates with a Pherastar plate-reader (635-20/680-20 nm, 10×10 matrix, 5 mm diameter, bottom-optic, gain=2000, focal height=1.7 mm) after various steps in the procedure. Plots and paired T tests were done with GraphPad Prism software. Fluorescent intensity was measured in arbitrary units. All errors displayed were standard error of the mean (SEM).

1. Method for covalent immobilization of molecular compounds on a substrate surface, comprising the steps:
a) Providing a substrate surface;
b) Treating the substrate surface with a plasma at atmospheric pressure, thereby generating at least one activated surface site;
c) Exposing at least a portion of the at least one activated surface site to molecular compounds, thereby establishing a covalent bond between the molecular compound and the substrate surface.

2. The method according to claim 1, wherein the substrate surface comprises a polymer material, or a polymerizable material which may preferably be deposited on the surface of a non-polymeric material such as a ceramic, semiconductor or metal.

3. The method according to claim 1, wherein the at least one activated surface site at least temporarily comprises radical species, preferably oxygen centered radicals, or reactive species.

4. The method according to claim 2, wherein the polymer material or polymerizable material is selected from a hydrocarbon polymer, such as polyethylene, polypropylene or polystyrene or precursors thereof, or from a heteroatom containing organic polymer, such as polytetrafluoroethylene, polyvinylchloride, polycaprolactam, polycaprolactone, poly(methyl)acrylate, polyethers or polyesters or precursors thereof.

5. The method according to claim 1, wherein the molecular compounds comprise cells, proteins, peptides, hydrogels, DNA, RNA, oligonucleotides, aptamers or antibiotics.

6. The method according to claim 1, wherein step b) is performed for 0.001 to 900 s, preferably 1 to 900 s, more preferably 1 to 10 s at a particular surface site.

7. The method according to claim 1, wherein step b) is repeated multiple times at a particular surface site, preferably 1 to 50 times, more preferably 5 to 20 times.

8. The method according to claim 1, wherein the plasma is generated with a plasma generation system comprising a nozzle and a moveable single electrode or a movable double-electrode.

9. The method according to claim 8, wherein the electrode is operated at a voltage of 1 to 25 kV, preferably 3 to 12 kV and/or at a frequency of 1 kHz to 10 GHz, preferably at 20 kHz to 40 kHz.

10. The method according to claim 1, wherein a distance of the nozzle to the substrate surface is between 0.1 to 200 mm, preferably between 1 and 10 mm.

11. The method according to claim 1, wherein step c) is performed for 5 minutes to 48 hours, preferably for 1 hour to 24 hours and/or wherein step c) is performed by 3D printing of the molecular compounds, or by depositing the molecular compounds by dropping or spraying.

12. The method according to claim 1, wherein a working gas is employed during step b), which is applied towards the substrate surface with a flow rate of at least 0.1 L/min.

13. The method according to claim 8, wherein the voltage and/or the flowrate are chosen such that a water contact angle at the activated surface site of 35° to 80° is obtained, when measured according to a contact angle test.

14. The method according to claim 1, wherein the molecular compounds are configured for adhesion of cells and/or signaling to cells and wherein the method further comprises the application of cells to the covalently immobilized molecular compounds.

15. The method according to claim 14, wherein the molecular compounds are proteins, preferably proteins which are configured for binding to the cell membrane or interacting with the cell membrane.

16. The method according to claim 14, further comprising the step of applying cells to the immobilized molecular compounds.

17. The method according to claim 1, wherein a predetermined pattern of immobilized molecular compounds is generated on the substrate surface by either
i) exposing only one or more predetermined portions in step c) to molecular compounds; or by
ii) treating only one or more predetermined sites of the substrate surface with the plasma in step b), thereby generating a predetermined pattern of at least one activated surface site.

18. A substrate comprising a substrate surface with covalently immobilized molecular compounds obtained with the method according to claim 1, wherein a water contact angle at the at least one activated surface site of 35° to 80° is obtained, when measured according to a contact angle test.
